(19) United States
(12) Patent Application Publication
(10) Pub. No.: US 2018/0000116 A1
(43) Pub. Date: Jan. 4, 2018

(54) METHOD FOR OBTAINING A PEPTIDE ISOLATE FROM A BIOMASS OF PROTEIN-ENRICHED MICROALGAE

(71) Applicant: Roquette Freres, Lestrem (FR)

(72) Inventors: Marilyne Guillemant, Aire sur la Lys (FR); Samuel Patinier, Quesnoy sur Deule (FR); Philippe Looten, Lomme (FR)

(73) Assignee: Roquette Freres, Lestrem (FR)

(21) Appl. No.: 15/546,206

(22) PCT Filed: Jan. 25, 2016
(86) PCT No.: PCT/FR2016/050139

§ 371 Date: Jul. 25, 2017

(30) Foreign Application Priority Data
Jan. 26, 2015 (FR) 1550569

(51) Int. Cl. A23J 1/00; A23L 27/23; C07K 14/405; A23J 3/34; A23L 33/18; A23L 27/00; C07K 1/36; C12N 1/12; C12P 21/00

(52) U.S. Cl. CPC A23J 1/009; A23L 33/18; A23L 27/235; C07K 14/405; A23L 27/88; A23J 3/347; C12N 1/12; C12P 21/00; C07K 1/36

(57) ABSTRACT

The invention relates to a peptide isolate isolated from a biomass of protein-rich microalgae, characterized in that it comprises:
soluble peptides with a molecular weight of between 1 and 20 kDa,
a protein content expressed as N.6.25 of more than 95%,
essentially arginine and glutamic acid.

The present invention relates to a peptide isolate derived from a biomass of protein-rich microalgae, and to microalgae of the Chlorella genus, even more particularly of the species Chlorella protothecoides.

Macroalgae and microalgae have a specific richness which remains largely unexplored. Their utilization for dietary, chemical or bioenergy purposes is still highly marginal. However, they contain components of great value, in terms of both richness and abundance.

Indeed, microalgae are sources of vitamins, lipids, proteins, sugars, pigments and antioxidants.

Algae and microalgae are thus of interest to the industrial sector, where they are used for manufacturing food supplements, functional foods, cosmetics and medicaments, or for aquaculture.

Microalgae are first and foremost photosynthetic microorganisms which colonize all biotopes exposed to light.

On the industrial scale, the monoclonal culturing thereof is performed in photobioreactors (autotrophic conditions: in light with CO₂) or, for some, it is also performed in fermenters (heterotrophic conditions: in darkness in the presence of a source of carbon).

This is because some species of microalgae are able to grow in the absence of light: Chlorella, Nitzschia, Cyclotella, Tetraselmis, Crypthecodinium, Schizochytrium.

Moreover, it is estimated that culturing under heterotrophic conditions is 10 times less expensive than under phototrophic conditions because, for those skilled in the art, these heterotrophic conditions allow:

the use of fermenters identical to those used for bacteria and yeast, enabling all the culturing parameters to be controlled,

the production of biomasses in much greater amounts than those obtained by light-based culturing.

The profitable utilization of microalgae generally necessitates controlling the fermentation conditions, making it possible to accumulate their components of interest, such as:

pigments (chlorophyll a, b and c, β-carotene, astaxanthin, lutein, phycocyanin, xanthophylls, phycoerythrin, etc.), the demand for which is increasing both due to their noteworthy antioxidant properties and to their provision of natural colorings for food,

lipids, in order to optimize their content of fatty acids (up to 60%, or even 80% by weight of their solids), especially for:
biofuel applications, but also
applications in food for human consumption or animal feed, when the selected microalgae produce “essential” (i.e. supplied by the diet because they are not naturally produced by humans or animals) polyunsaturated fatty acids or PUFAs, or

proteins, in order to optimize the nutritive qualities thereof or, for example, to promote the supply of amino acids of interest by means of preparing peptide-rich fractions.

The peptide fractions may be upgraded as functional agents or food supplements in many fields.

In the context of supplying amino acids of interest, it may in fact be advantageous to have available peptide sources that are rich in arginine and glutamic acid.

Arginine is an amino acid that has many functions in the animal kingdom.

Arginine may be degraded and may thus serve as a source of energy, carbon and nitrogen for the cell which assimilates it.

In various animals, including mammals, arginine is decomposed into ornithine and urea. The latter is a nitrogenous molecule that can be eliminated (via excretion in the urine) so as to regulate the amount of nitrogenous compounds present in the cells of animal organisms.

Arginine allows the synthesis of nitrogen monoxide (NO) via NO synthetase, thus participating in the vasodilation of the arteries, which reduces the rigidity of the blood vessels, increases the blood flow and thus improves the functioning of the blood vessels.

Food supplements which contain arginine are recommended for promoting the health of the heart, the vascular function, for preventing “platelet aggregation” (risk of formation of blood clots) and for lowering the arterial pressure.

The involvement of arginine in the healing of wounds is associated with its role in the formation of proline, which is another important amino acid in collagen synthesis.

Finally, arginine is a component that is frequently used, in particular by sportspeople, in energy drinks.

As regards glutamic acid, it is not only one of the elementary bricks used for protein synthesis, but is also the excitatory neurotransmitter that is the most widespread in the central nervous system (encephalon+spinal column) and is a GABA precursor in GABAergic neurons.

Under the code E620, glutamate is used as a flavor enhancer in foods. It is added to food preparations to enhance their taste.

Besides glutamate, the Codex Alimentarius has also recognized as flavor enhancers the sodium salt (E621), the potassium salt (E622), the calcium salt (E623), the ammonium salt (E624) and the magnesium salt (E625) thereof.

Glutamate (or the salts thereof) is often present in ready-made meals (soups, sauces, crisps and ready-made dishes). It is also commonly used in Asian cookery.

It is currently frequently used in combination with flavorings in aperitifs (bacon flavor, cheese flavor). This makes it possible to enhance the bacon, cheese, etc. flavor. It is rare to find an aperitif not containing any.

It is also found in certain medicament capsules, but not for its taste functions.

Finally, it is the major component of cooking auxiliaries (stock cubes, sauce bases, sauces, etc.).

However, the developments in food applications of microalgal peptide fractions have not been significant due to the presence in said fractions of undesirable compounds (pigments, etc.). These compounds lead to undesired changes in color, taste and structure of the food compositions containing them.

To increase their potentiality in food applications and to increase also their commercial value, these peptides must thus be extracted from microalgae having the required compositions, in terms of:
richness in nitrogen,
richness in amino acids of interest,
low content of pigments.

### SUBJECT OF THE INVENTION

The present invention relates to a peptide isolate derived from a biomass of protein-rich microalgae, and to microalgae of the Chlorella genus, even more particularly of the species Chlorella protothecoides.

More particularly, the present invention relates to a microalgal isolate characterized by its remarkably high content of arginine and of glutamic acid.

The present invention also relates to the biomass of protein-rich microalgae per se, this biomass being particularly suitable for preparing said peptide isolate.

The present invention also relates to the method for enriching and depigmenting a biomass of microalgae, more particularly of the Chlorella genus, even more particularly of the species Chlorella protothecoides.

Finally, the present invention relates to the method for preparing this peptide isolate from the biomass of protein-rich and depigmented microalgae.

Specifically, in order to be able to exploit the metabolic richnesses of microalgae, and more particularly their peptide fractions, the Applicant company proposes to provide a peptide isolate having:
soluble peptides with a molecular weight of between 1 and 20 kDa,
a protein content expressed as N.6.25 of more than 95%,
essentially arginine and glutamic acid.

For the purposes of the invention, the expression “essentially composed of arginine and glutamic acid” means a richness in arginine and glutamic acid which may be understood as a content of arginine and glutamic acid of more than 80, 85, 90 or 95% by weight expressed relative to the total amino acids. In particular, these two amino acids represent 85 to 99% relative to the total amino acids, preferably between 90 between 98% relative to the total amino acids, and in particular between 95 and 98%.

More precisely, for the purposes of the invention, the expression “essentially composed of arginine and glutamic acid” means a richness in arginine and glutamic acid which may be understood as a content:
of at least 40% of arginine, especially between 40% and 60%, preferably about 50%; and
of at least 40% of glutamic acid, especially between 40% and 60%, preferably about 50%

expressed relative to the total amino acids.

In particular, the content of amino acids other than arginine and glutamic acid is less than 10%, preferably less than 5%, especially less than 3%.

In one specific embodiment, the isolate content is as follows:
a content of between 47 and 48% of arginine;
a content of between 49 to 50% of glutamic acid;

expressed relative to the total amino acids, which is reflected by a content of amino acids other than arginine and glutamic acid of less than 3%.

The term “approximately” is intended to mean the value range comprising plus or minus 10% of the indicated value, preferably plus or minus 5% thereof. For example, “approximately 10” means between 9 and 11, preferably between 9.5 and 10.5.

This peptide isolate may be prepared from a biomass of microalgae of the genus Chlorella, even more particularly of the species Chlorella protothecoides, decolorized microalgae having a protein content, expressed as N.6.25, of greater than 60%, for example more than 65%.

The preferred method for fermenting microalgae is a two-step method, comprising:
a first fermentation step, deficient in nitrogen, in which the pH regulation is performed with an NH₃/KOH mixture, and then
a second step of removal of this nitrogen deficiency by a pH regulation performed with NH₃ alone.

These operating conditions thus make it possible rapidly to obtain a biomass with a protein content of greater than 60% of N.6.25, of the order of or about 65% of N.6.25, and low coloration. The yield is from 45 to 50% by weight of solids, and the final concentration of biomass is between 80 and 120 g/l.

Moreover, the content of residual salts of the soluble fraction of the fermentation must does not exceed 6 g/l.

The biomass thus prepared is then washed to purify it of its interstitial soluble substances (especially soluble salts), brought to a solids content of between 15 and 30%, preferably to a solids content of between 20 and 30%, and then heat-treated at a temperature of between 50 and 150° C. for a time of between 5 seconds and 5 minutes.

On conclusion of this treatment, which permeabilizes the cell membrane and allows the release of the soluble components of the intracellular compartment by free diffusion, the residual biomass is removed, and the soluble fraction recovered is then clarified, precipitated, concentrated and then dried to constitute the peptide isolate in accordance with the invention.

The present invention thus relates to an isolate that is obtained or that may be obtained from a biomass of protein-rich microalgae prepared via a fermentation method described in the present document. The invention also relates to an isolate that is obtained or that may be obtained from the biomass of protein-rich microalgae via a method for treating the biomass as described in the present document.

### DETAILED DESCRIPTION OF THE INVENTION

### Characterization of the Peptide Isolate According to the Invention

The invention relates to a peptide isolate prepared from a biomass of microalgae cultivated so as to enrich it in protein, the microalgae being derived from the genus Chlorella, more particularly Chlorella protothecoides.

The peptide isolate in accordance with the invention, obtained from this protein-rich biomass, is characterized in that it comprises:
soluble peptides with a molecular weight of between 1 and 20 kDa,
a protein content expressed as N.6.25 of more than 95%,
essentially arginine and glutamic acid.

In this context of definition of the isolate, the term “comprises” means that the isolate is formed essentially by these peptides, but little comprise other minor components. Thus, the term “formed essentially” means at least 90, 95 or 99% by dry weight of the isolate.

The molecular weight of said peptides is measured by chromatography according to the following method:

Chromatographic conditions:
2 columns mounted in series: SUPERDEX® 200HR10/30 column with a SUPERDEX® Peptide HR10/30 column (from Pharmacia Biotech)
Flow rate: 0.3 ml/min
UV detector at 214 nm
Eluent: NaCI 0.05 M
Analysis time: 240 min

The sample is dissolved at 0.5% in HPLC-grade water.

The columns are calibrated with a Biorad control mixture ref. 151-1901 composed of:
Thyroglubulin: Mw=670 KDa
Bovine globulin: Mw=158 KDa
Ovalbumin: Mw=44 KDa
Myoglobin: Mw=17 KDa
Vitamin B12: Mw=1.35 KDa

The percentage of the various fractions is then calculated on the basis of the retention times of each control.

Measurement of the protein content is conventionally determined by measuring the N.6.25, which is generally known.

Finally, the amino acid composition is determined according to NF EN ISO 13903 (November 2005).

The arginine and glutamic acid contents of the isolate are as stated previously in this document.

The high content of arginine and glutamic acid is understood herein, for example, to mean a content:
of between 47 and 48% of arginine
of between 49 to 50% of glutamic acid

expressed relative to the total amino acids, which is reflected by a content of amino acids other than arginine and glutamic acid of less than 3%.

Optionally, the peptide isolate comprises less than 3% of total sugars (carbohydrates).

### Method for Preparing the Peptide Isolate in Accordance with the Invention

The peptide isolate in accordance with the invention may be prepared from a biomass of microalgae of the genus Chlorella, even more particularly of the species Chlorella protothecoides, decolorized microalgae having a protein content, expressed as N.6.25, of greater than 60%.

To obtain maximum productivity and yields of protein, the Applicant company used a novel method which it has protected elsewhere in one of its recently filed patent applications.

In the prior art, first fermentation methods for obtaining high cell densities (abbreviated as HCD) were extensively studied.

The aim of these HCD cultures was to obtain the highest possible concentration of the desired product in the shortest possible period of time.

However, maintaining growth at its maximum rate (μ, in h⁻¹) is not always correlated with high production of the product of interest.

Consequently, in the event that the formation of products is not correlated with high or maximum cell growth, it is prudent to control the rate of cell growth.

In general, those skilled in the art choose to control the growth of the microalgae by controlling the fermentation conditions (temperature, pH) or by regulated feeding of nutritional components (nitrogen or carbon sources) to the fermentation medium, under semicontinuous conditions referred to as “fed batch”.

Indeed, Chlorella protothecoides is acknowledged to be one of the best oil-producing microalgae.

Under heterotrophic conditions, it rapidly converts carbohydrates to triglycerides (more than 50% of the solids thereof).

To optimize this production of triglycerides, those skilled in the art are led to optimize the carbon flow toward oil production, by acting on the nutritional environment of the fermentation medium.

Thus, it is known that oil accumulates when there is a sufficient supply of carbon but under conditions of nitrogen deficiency.

Therefore, the C/N ratio is the determining factor here, and it is accepted that the best results are obtained by acting directly on the nitrogen content, with the glucose content not being a limiting factor.

However, Chlorella protothecoides may also be used for its capacity to produce protein.

For the production of protein-rich biomasses, those skilled in the art are therefore led to perform the opposite of metabolic control for allowing the microalga naturally to produce storage lipids, i.e. to modify the fermentation conditions by instead promoting a low C/N ratio, and thus:
to supply a large amount of nitrogen source to the fermentation medium while keeping constant the carbon source feedstock, which will be converted into protein, and
to stimulate the growth of the microalga.

This involves modifying the carbon flow toward protein (and hence biomass) production, to the detriment of storage lipid production.

In the context of the invention, the Applicant company has, on the other hand, chosen to explore a novel route by proposing alternative solutions to those conventionally envisioned by a person skilled in the art.

The method for the heterotrophic culturing of said microalgae developed by the Applicant company to increase the protein content of biomass then comprises:
a “batch” fermentation phase characterized, after seeding of the fermenter, by supplying in a single portion an amount of glucose of between 15 and 25 g/l, preferably about 20 g/l,
an exponential fed batch phase during which glucose is supplied gradually and the pH regulation performed initially by the NH₃/KOH mixture is replaced by regulation with NH₃ alone.

As will be illustrated below, supplying NH₃ induces a remarkably rapid increase in the level of protein synthesized in the cell, which is reflected by an increase in the level of intracellular N.6.25 to a value exceeding 60%.

Full analysis of the amino acids present in the biomass was then performed on a sample taken just before changing the pH regulation, and on several other samples taken after said change.

It is observed that, before the change, the sum of the amino acids is low (of the order of 15 to 25%) and that there is no predominance among the various amino acids.

After the regulation change, it is noted that:
the sum of the amino acids exceeds 40%,
in total, the content of glutamic acid and arginine relative to the total amino acids is more than 45%,
the amino acid which undergoes the greatest increase is glutamic acid, followed by arginine. The content of the other amino acids also increases, but to a much lower extent.

The increase in the N.6.25 is thus directly correlated with the increase in glutamic acid and arginine synthesis.

Moreover, this pH regulation method makes it possible:
to limit the salt supply of the fermentation medium, and
to modify the color of the biomass, which will be proportionately yellower the more the initial content of NH3 is limited.

In conclusion, the biomass of protein-rich microalgae, the microalgae being of the genus Chlorella, even more particularly of the species Chlorella protothecoides, has:
a concentration of 80 to 90 g/l,
an N.6.25 content of more than 60%,
an amount of salts of less than 6 g/l in its culture supernatant,
a low coloration, and
a content of amino acid and glutamine of more than 45% by weight relative to the total amino acids.

This biomass is particularly suitable for preparing the peptide isolate according to the invention, by performing the following method:
optionally, washing the biomass so as to remove the interstitial soluble compounds,
thermal permeabilization of the biomass at a temperature of between 50 and 150° C., preferably between about 80 and 150° C., for a time of between about 10 seconds and about 5 minutes, preferably for a time of between about 10 seconds and about 1 minute,
removal of the biomass permeabilized in this way by a technique of solid-liquid separation, preferably chosen from the group formed by frontal or tangential filtration, flocculation and centrifugation, more particularly multistage centrifugation, to obtain a soluble fraction,
optionally, recovery and clarification of the soluble fraction obtained in this way by microfiltration so as to free it of residual insoluble substances,
purification of the soluble fraction by precipitation, so as to obtain a peptide isolate, and
evaporation, pasteurization and atomization of said peptide isolate.

After fermentation under the conditions listed above, the biomass is collected by solid-liquid separation, by frontal or tangential filtration or by any means additionally known to those skilled in the art.

Optionally, the Applicant company recommends washing the biomass in such a way as to remove the interstitial soluble compounds by a succession of concentration (by centrifugation)/dilution of the biomass.

For the purposes of the invention, the term “interstitial soluble compounds” means all the soluble organic contaminants of the fermentation medium, for example the water-soluble compounds such as the soluble salts, the residual glucose, the oligosaccharides with a degree of polymerization (or DP) of 2 or 3, or the peptides.

This biomass purified in this way of its interstitial soluble compounds is then preferentially adjusted to a solids content of between 15 and 30% by weight, preferably to a solids content of between 20 and 30%.

The heat treatment is performed at a temperature of between 50 and 150° C., preferably between about 80 and 150° C., for a time of between about 5 seconds and about 5 minutes, preferably for a time of between about 10 seconds and about 1 minute. Preferably, the heat treatment is performed at a temperature of about 140° C., for a time of about 10 seconds. In another preferred alternative, the heat treatment is performed at a temperature of about 85° C., for a time of about 1 minute.

This treatment makes it possible to allow the intracellular components to diffuse into the reaction medium.

Finally, at the end of these steps, the biomass is cooled to a temperature of below 40° C., preferably refrigerated at a temperature of the order of 4° C.

Without wishing to be bound by a particular theory, the Applicant company considers that the thermal treatment, performed under these operating conditions, could thus act as a membrane weakening process which allows the spontaneous release of the soluble components of the intracellular compartment, or even of the extracellular matrix.

In addition to the ionic substances, organic substances such as carbohydrates (predominantly DP1 and DP2), the peptides and the polypeptides are drained out of the cell.

Conversely, the lipids and hydrophobic organic compounds remain in the cells, thereby clearly demonstrating that the cells are permeabilized and not lyzed or destroyed.

The method according to the invention does not therefore result in the formation of an emulsion, but indeed of an aqueous suspension.

The release of all these soluble substances through the permeabilized membrane is similar to a process of free diffusion of dialysis type.

Consequently, a lag time may be necessary in order to allow sufficient diffusion after the heat treatment which permeabilizes the membrane.

In the literature, the process for pulsed-field permeabilization of yeast membranes in order to extract the proteins therefrom requires from 4 h to 6 h (Ganeva et al., 2003, Analytical Biochemistry, 315, 77-84).

According to the invention, a much shorter reaction time is used, of between 5 seconds and 5 minutes.

Raising the scale (time/temperature) then leads to an increase in the degree of dissolution and in the yield of soluble matter extraction.

The method of the invention advantageously exploits the phenomenon of thermal permeabilization to extract the peptide fraction thus dissolved from the residual biomass.

Thus, the residual biomass is then removed by a technique of solid-liquid separation by frontal or tangential filtration, by flocculation, by centrifugation or by any means additionally known to those skilled in the art, thereby making it possible easily to recover the soluble fraction freed of the microalgal cells.

The yield and quality of this separation step may be improved by diluting the permeabilized cells (for example by dilution/multistage centrifugation).

If necessary, the soluble fraction thus obtained may be clarified by microfiltration so as to free it of the residual insoluble matter and, depending on its solids content, a concentration by evaporation or by any other means additionally known to those skilled in the art may be performed before the purification that follows.

The resulting soluble fraction is finally essentially composed of protein (50-80% w/w) and carbohydrates (10-25% w/w).

The conventional methods for recovering soluble proteins are generally based on a step of precipitating said proteins with trichloroacetic acid (10% weight/volume) or with ammonium sulfate.

However, these isolations by precipitation follow on from very destructive cell-breaking methods (usually by sonication or homogenization) which, while they make it possible in fact to increase extraction yields, result especially in proteins of low solubility which are denatured.

It is then possible to envision the refunctionalization thereof only by means of their product of hydrolysis (to peptides) by chemical means (lysis with sodium hydroxide), physical means (high-temperature treatment) or enzymatic means (proteolytic enzymes).

The method of the invention then leads to isolation of the proteins of interest, by precipitation by modifying the properties of the medium.

The Applicant company thus recommends proceeding as follows:
promoting the precipitation of all or part of the protein fraction by modifying the physicochemical properties of the medium.
The cooling of the crude soluble matter, obtained as described in the preceding steps, triggers a phenomenon of precipitation of part of the soluble peptides. It is observed that the precipitation is rather selective toward the higher molecular weights. The cooling temperature is below 10° C., preferably below 4° C.
Certain operating conditions make it possible to promote this phenomenon: besides the temperature, the pH must be between 2.5 and 6.5 and preferably close to the pHi, i.e. between 3 and 5.
Similarly, the ionic strength of the medium may be adapted to promote precipitation. Thus, by greatly reducing the ionic strength, the phenomenon of “salting-in” may be attenuated, and the solubility of the proteins may thus be reduced (by reducing the solvation layer). Thus, a demineralization operation prior to the precipitation may be added. This is performed on cationic and anionic resins, dialysis, filtration or by any means additionally known to those skilled in the art. Conversely, by greatly increasing the ionic strength, the available water decreases via the phenomenon of “salting-out”, and in this way the proteins have a tendency to precipitate. This method is not preferred since pronounced demineralization would then be necessary on the protein isolate thus extracted. In the same perspective of modulating the solvation layer, the polarity of the medium may be reduced (with dehydration of the medium) by adding a solvent such as ethanol which will make it possible to generate more quantitative precipitation of the protein fraction by greatly reducing its solubility.
by recovering the precipitated fraction which is then optionally concentrated before drying.
Separation of the precipitated fraction is performed by simple decantation and recovery of the heavy phase or optionally by centrifugation under optimum temperature conditions.
The pH may optionally be readjusted before drying.
Drying is performed by atomization, lyophilization or by any other means additionally known to those skilled in the art.
Prior to drying, the incorporation of a step of concentration by evaporation may make it possible to optimize the operation in energy terms. It may especially be justified if a solvent such as ethanol is used to perform its recycling.

Exploiting these approaches makes it possible to purify a fraction with a high content of peptides and polypeptides from the residual salts and sugars.

A soluble protein isolate is then obtained at greater than 90% by weight, which is rich in arginine and glutamic acid.

The invention will be understood more clearly from the following examples which are intended to be illustrative and nonlimiting.

### EXAMPLES

### Example 1

### Preparation of a Biomass of C Protothecoides Rich in Protein with a High Content of Glutamic Acid and Arginine

The strain used is a Chlorella protothecoides (strain CCAP211/8D—The Culture Collection of Algae and Protozoa, Scotland, UK).

Preculture:
150 mL of medium in a 500 mL conical flask;
Composition of the medium: 40 g/L of glucose+10 g/L of yeast extract. Incubation is performed under the following conditions:
time: 72 h;
temperature: 28° C.;
shaking: 110 rpm (Infors Multitron Incubator).

Culturing in Batch and Then Fed Batch Mode

Preparation and Initial Batch Medium
prepare and filter a mixture of KOH at 400 g/l (41%)/NH3 at 20% v/v (59%);
sterilize 20 L fermenter at 121° C./20 min;
inoculate with 2 conical flasks of 500 mL of preculture (OD_{600 nm}^{of} 15);
brought to pH 4.5 with 20% (v/v) NH₃OH
starting shaking speed of 300 rpm;
aeration: 20 L/min of air;
pO₂ regulation at 30%;

Feed
glucose: 500 g/L
ammonium sulfate: 5 g/L
diammonium phosphate: 20 g/L
phosphoric acid: 16 g/L
magnesium sulfate heptahydrate: 12 g/L
iron sulfate: 170 mg/L
calcium nitrate: 610 mg/L
solution of trace elements: 45 mL/L
solution of vitamins: 4.5 mL/L

It is important to note that the feedstock of ammonium salts, magnesium salts and phosphoric acid was developed so as to limit the salt content of the fermentation medium and was optimized so as to maintain the N.6.25 content of the final decolorized biomass.

Solution of trace elements

| Ingredients | (g/l) |
|---|---|
| CuSO₄ | 0.22 |
| ZnSO₄ | 7 |
| MnSO₄ | 4 |
| Citric acid | 30 |

Solution of vitamins

| Ingredients | (g/l) |
|---|---|
| Thiamine HCl | 13.4 |
| Biotin | 0.2 |
| B12 | 0.16 |
| Calcium pantothenate | 0.4 |
| p-Aminobenzoic acid | 0.8 |

Fermentation Procedure
provide the equivalent of 20 g/L before inoculation
when the glucose concentration is=0 g/L, start the feed in exponential profile (μ=0.07 h⁻¹);
regulate the pH at 5.2 with a 41% KOH/59% NH₃ mixture
when 2 kg of glucose have been consumed by the microalga, switch the system to pH regulation with NH₃ alone

Results:

This fermentation procedure makes it possible to obtain a biomass with more than 65% protein, expressed as N.6.25.

### Example 2

### Method for Thermal Permeabilization of Protein Enriched Biomass and Recovery of the Crude Soluble Matter

The biomass obtained according to Example 1 is harvested at a cell solids content of 105 g/L with a purity of 80% (purity defined by the ratio of the solids content of the biomass to the total solids content).

It is then:
washed and concentrated by inline dilution [1:1] (V_{water}Vₘᵤₛₜ),
centrifuged on an Alfa Laval FEUX 510 plate centrifuge and brought to a solids content of 220 g/l and to a purity of 93% (purity defined by the ratio of the solids content of the biomass to the total solids content).

The pH is adjusted to 7 with potassium hydroxide and the biomass is heat-treated by UHT with preheating at 70° C. followed by direct injection of steam on a scale of about 10 seconds at 140° C. and flash cooling to 40° C. under vacuum.

The heat treatment is pushed to a high scale so as to maximize the partial dissolution of the biomass, the purity of which decreases to 53%.

By definition, the salting-out of the soluble matter in the extracellular medium leads to a decrease in the fraction of cell solids relative to the total solids content.

At this stage, the composition of the biomass is as follows:
Total amino acids: 48.4%
Total sugars: 27.2%
Total fatty acids: 15.1%
Ash: 2.5%
Other residues: 6.8%

Separation of the soluble matter derived from the salting-out by thermal permeabilization of biomass is performed by centrifugal separation.

In order to optimize the separation yield and quality, a slight dilution [0.5:1] (V_{water}Vₘᵤₛₜ) is performed inline on the second stage (on a configuration with two Alfa Laval FEUX 510 centrifuges in series) with recycling of the supernatant from the second stage into the first.

The supernatant from the first stage is thus recovered and the clarified soluble matter is concentrated.

This “crude” soluble matter has the following composition:
Total amino acids: 77.3%
Total sugars: 17.6%
Ash: 4.3%
Other residues: 0.8%

### Example 3

### Purification and Production of the Protein Isolate

In order to selectively precipitate the protein fraction, 5 kg of crude soluble matter with a solids content of 11.4% are placed in a jacketed reactor with stirring.

The pH of the crude soluble matter is adjusted to 4.5 with phosphoric acid.

After stopping the stirring, the temperature is lowered to 4° C.

These conditions are maintained for 8 hours.

Decantation of the heavy phase enriched in peptides of higher molecular weight is thus performed.

The heavy phase is then extracted by simple phase separation in a separating funnel, with a mass yield of 26% and has a solids content of 36.3%.

This extract is lyophilized to a solids content of 97%.

The composition of this isolate is as follows:
Total amino acids: 95.9%
Total sugars: 2.44%
Ash: 1.66%

Analysis of the amino acid distribution in the total amino acids is as follows:
glutamic acid: 49.78%
arginine: 47.21%
other amino acids: 3.01%

The isolate is characterized by a richness of the order of 95% of amino acids formed essentially by arginine and glutamic acid (on the basis of the distribution analysis of the total amino acids).

The molecular weight of this fraction is essentially between 1 kDa and 20 kDa.

1. A peptide isolate isolated from a biomass of protein-rich microalgae, said isolate comprising:
soluble peptides with a molecular weight of between 1 and 20 kDa,
a protein content expressed as N.6.25 of more than 95%, wherein total amino acid content of said protein substantially comprises arginine and glutamic acid.

2. The isolate according to claim 1, wherein the microalgae are of genus Chlorella.

3. The isolate according to claim 1, wherein the content of arginine and glutamic acid is more than 80% by weight expressed relative to the total amino acids.

4. The isolate according to claim 1, wherein the content of arginine and glutamic acid is respectively at least 40% exppresse relative to the total amino acid content.

5. The isolate according to claim 4, wherein the content of arginine is between 47 and 48% for arginine and between 49 to 50% for glutamic acid expressed relative to the total amino acid content.

6. The isolate according to claim 1, prepared by fermentation of a biomass of protein-rich microalgae genus Chlorella method which comprises:
a “batch” fermentation phase in a fermenter of the biomass of protein-rich microalgae genus Chlorella after seeding the fermenter, by supplying in a single portion an amount of glucose of between 15 and 25 g/l,
an exponential fed batch phase during which glucose is supplied gradually and pH regulation is performed initially by a NH₃/KOH mixture and subsequently replaced by regulation with NH₃ alone.

7. The isolate according to claim 1, obtained from a biomass of protein-rich microalgae of genus Chlorella, by a method which comprises:
optionally, washing the biomass so as to remove the interstitial soluble compounds,
thermal permeabilization of the biomass at a temperature of between 50 and 150° C., for a time of between about 10 seconds and about 5 minutes,
removal of the biomass permeabilized in this way by a technique of solid-liquid separation including one of frontal or tangential filtration, flocculation and centrifugation to obtain a soluble fraction,
optionally, recovery and clarification of the soluble fraction obtained to free it of residual insoluble substances,
purification of the soluble fraction by precipitation, so as to obtain the peptide isolate, and
evaporation, pasteurization and atomization of said protein isolate.

8. The isolate according to claim 1, wherein the microalgae comprise Chlorella protothecoides.

9. The isolate according to claim 1, wherein the content of arginine and glutamic acid is more than 95% by weight expressed relative to the total amino acids.

10. A peptide isolate isolated from a biomass of protein-rich microalgae, said isolate comprising:
a soluble peptides with a molecular weight of between 1 and 20 kDa, a protein content expressed as N.6.25 of more than 95%, wherein the total amino acid content of said protein substantially comprises arginine and glutamic acid, and amino acid contents other than arginine and glutamic acid amount to less than 3%.
